(19) United States
(12) Patent Application Publication
(10) Pub. No.: US 2026/0314910 A1
(43) Pub. Date: Oct. 15, 2026

(54) JOYSTICK MECHANISM, OPERATING HANDLE AND AN ENDOSCOPE

(71) Applicant: ScopeAround, Irvine, CA (US)

(72) Inventor: Yingjie SUN, Irvine, CA (US)

(73) Assignee: ScopeAround, Irvine, CA (US)

(21) Appl. No.: 19/216,357

(22) Filed: May 22, 2025

(30) Foreign Application Priority Data
Apr. 14, 2025 (CN) 202520700465.1
Apr. 14, 2025 (CN) 202520703679.4

(51) Int. Cl. A61B 1/00; A61B 34/00

(52) U.S. Cl. CPC A61B 1/0004; A61B 34/74; A61B 2034/742

(57) ABSTRACT

The present disclosure provides a joystick mechanism, an operating handle and an endoscope. The endoscope includes an operating handle, a display screen and a snap-fit assembly, the display screen and the operating handle are detachably connected. The joystick mechanism includes a mounting seat, an outer sleeve, a support column, a head joint, a first elastic member, and a joystick assembly.

### CROSS-REFERENCE TO RELATED APPLICATIONS

This application claims priority benefits to Chinese Patent Application No. 2025207004651, filed on Apr. 14, 2025, and Chinese Patent Application No. 2025207036794, filed on Apr. 14, 2025, the contents of which are both incorporated herein by reference.

### TECHNICAL FIELD

The present disclosure relates to the technical field of endoscopes, and in particular, to a joystick mechanism, an operating handle and an endoscope.

### BACKGROUND

Industrial endoscopes, as a crucial inspection tool in modern engineering operations, enable the examination of concealed or visually inaccessible areas without requiring large-scale disassembly or causing damage to target objects. Typically equipped with an operating handle, these devices feature a joystick mechanism on the handle. This joystick controls the bending direction of the distal end of the endoscope, thereby directing its insertion path and observation orientation.

### SUMMARY

A first aspect of the present disclosure provided a joystick mechanism, including: a mounting seat; an outer sleeve connected to the mounting seat, and one end of the outer sleeve being provided with a first retention portion; a support column disposed in a cavity of the outer sleeve, the support column configured to move along an axial direction of the cavity, and one end of the support column defining a first mounting groove; a head joint having a ball head, the ball head being disposed in the first mounting groove and secured between the support column and the first retention portion, the ball head configured to move in the first mounting groove, and an end of the head joint extending out of the outer sleeve; a first elastic member arranged in the cavity of the outer sleeve, a first end of the first elastic member being connected to the support column, a second end of the first elastic member being connected to the mounting seat, and the first elastic member configured to squeeze the support column to be away from the mounting seat; and a joystick assembly connected to a portion of the head joint, the portion of the head joint extending out of the outer sleeve, and the joystick assembly configured to control the head joint to move in the first mounting groove.

A second aspect of the present disclosure provides an operating handle, including the joystick mechanism described above, a lens assembly and a protective coupling assembly. A portion of the joystick mechanism is arranged in the housing and connected to an inner wall of the housing by the mounting seat, and the operating end of the operating rod extends out of the housing. The lens assembly is connected to the housing, a proximal end of the lens assembly extends into the housing and is connected to the linkage member, and a distal end of the lens assembly extends away from the housing. The protective coupling assembly is sleeved on the proximal end of the lens assembly and connected to the housing.

A third aspect of the present disclosure provides an endoscope, including: an operating handle provided with a connector, and a first connecting terminal is provided on the connector; a display screen detachably connected to the operating handle, and the display screen comprising a casing and a second connecting terminal, the casing defining a connecting groove, the connector being inserted into the connecting groove to detachably assemble the operating handle and the display screen, a fixed end of the second connecting terminal being assembled in an inner cavity of the casing, an insertion end of the second connecting terminal extending through the casing and extending into the connecting groove, and the second connecting terminal is configured to be plugged into the first connecting terminal to conduct the operating handle and the display screen, and a sidewall of the connecting groove defining a snap-fit channel communicating with the inner cavity of the casing; and a snap-fit assembly including a first portion and a second portion, the first portion of the snap-fit assembly being fixedly disposed in the inner cavity of the casing, the second portion of the snap-fit assembly being accommodated in the snap-fit channel and connected to the first portion of the snap-fit assembly, the second portion of the snap-fit assembly is configured to move relative to the snap-fit channel and extend out of the snap-fit channel to snap fit with the connector.

### BRIEF DESCRIPTION OF DRAWINGS

The drawings that form part of the present disclosure are provided to further illustrate the present disclosure. The illustrative embodiments and their descriptions of the present disclosure are intended to explain but do not constitute an undue limitation on the present disclosure.

FIG. 1 is a perspective view of an endoscope according to some embodiments of the present disclosure.

FIG. 2 is a perspective view of an operating handle according to some embodiments of the present disclosure.

FIG. 3 is a front view of FIG. 2.

FIG. 4 is a cross-sectional view taken along line A-A in FIG. 3.

FIG. 5 is an exploded view of an operating handle according to some embodiments of the present disclosure.

FIG. 6 is a perspective view of an operating handle according to some embodiments of the present disclosure.

FIG. 7 is a perspective view of a first housing according to some embodiments of the present disclosure.

FIG. 8 is a perspective view of a second housing according to some embodiments of the present disclosure.

FIG. 9 is a perspective view of a joystick mechanism, a protective coupling assembly and a lens tube according to some embodiments of the present disclosure.

FIG. 10 is a perspective view of a mounting seat, an outer sleeve, a support column, a head joint and a first elastic member according to some embodiments of the present disclosure.

FIG. 11 is a perspective view of a mounting seat, an outer sleeve, a support column, a head joint and a first elastic member according to some embodiments of the present disclosure.

FIG. 12 is a front view of an outer sleeve, a support column, a first elastic member and a mounting seat after assembly according to some embodiments of the present disclosure.

FIG. 13 is a cross-sectional view taken along the line B-B in FIG. 12.

FIG. 14 is a perspective view of a joystick mechanism and a traction line, a traction tube, a fixing member, and a fixing block according to some embodiments of the present disclosure.

FIG. 15 is a cross-sectional view taken along line C-C in FIG. 14.

FIG. 16 is an exploded view of a joystick assembly according to some embodiments of the present disclosure.

FIG. 17 is a perspective view of a linkage member according to some embodiments of the present disclosure.

FIG. 18 is a first exploded view of an endoscope according to some embodiments of the present disclosure.

FIG. 19 is a second exploded view of an endoscope according to some embodiments of the present disclosure.

FIG. 20 is a perspective view of a connector according to some embodiments of the present disclosure.

FIG. 21 is a perspective view of a display screen according to some embodiments of the present disclosure.

FIG. 22 is an enlarged view of area D in FIG. 21.

FIG. 23 is a left view of the display screen in FIG. 21.

FIG. 24 is a perspective view of a display screen according to some embodiments of the present disclosure.

FIG. 25 is a perspective view of a rear cover and the snap-fit assembly according to some embodiments of the present disclosure.

FIG. 26 is a perspective view of a rear cover and a snap-fit assembly according to some embodiments of the present disclosure.

FIG. 27 is a perspective view of a housing and a connector according to some embodiments of the present disclosure.

FIG. 28 is an enlarged view of area E in FIG. 27.

FIG. 29 is a perspective view of a housing and a connector according to some embodiments of the present disclosure.

FIG. 30 is an enlarged view of area F in FIG. 29.

FIG. 31 is a perspective view of a second housing, a protrusion, an extension and a cover according to some embodiments of the present disclosure.

FIG. 32 is a perspective view of an operating handle, a connector and a snap-fit assembly according to some embodiments of the present disclosure.

FIG. 33 is an exploded view of a snap-fit assembly according to some embodiments of the present disclosure.

FIG. 34 is a perspective view of a seat of a snap-fit assembly according to some embodiments of the present disclosure.

FIG. 35 is a perspective view of a snap-fit member of a snap-fit assembly according to some embodiments of the present disclosure.

### DETAILED DESCRIPTION OF THE EMBODIMENTS

The present disclosure is described in detail below with reference to the accompanying drawings and in conjunction with various embodiments. Each example is provided to explain but not to constitute undue limitation on the present disclosure. In fact, it is clear to those of ordinary skill that modifications and variations may be made without departing from the scope or spirit of the present disclosure. For example, a feature shown or described as a part of some embodiments may be used by some embodiments to produce yet some embodiments. Therefore, it is intended that the present disclosure includes such modifications and variations within the scope of the appended claims and their equivalents.

In the description of the present disclosure, the terms “longitudinal”, “lateral”, “upper”, “lower”, “front”, “back”, “left”, “right”, “vertical”, “horizontal”, “top”, “bottom” and the like indicate the orientational or positional relationships based on the orientational or positional relationships illustrated in the drawings, which are only for the convenience of describing and do not require the present disclosure to be constructed and operated in a specific orientation, and therefore shall not be understood as limitation on the present disclosure. The terms “connected”, “connecting” and “arranged” used in the present disclosure should be understood in a broad sense. For example, it may be a fixed connection or a detachable connection; it may be directly connected or indirectly connected through an intermediate component; it may also be a wired electrical connection, a radio connection, or a wireless signal connection. For those of ordinary skill in the art, the specific meanings of the above terms may be understood according to the specific circumstances.

One or more examples of the present disclosure are illustrated in the attached drawings. Characters and letters are used in the detailed description to refer to features in the drawings. Similar signs in the drawings and descriptions have been configured to refer to similar parts of the present disclosure. As used herein, the terms “first”, “second” and “third” are used interchangeably to distinguish one component from another and are not intended to indicate the position or importance of each component.

In the description of the embodiments of the present disclosure, in some embodiments, the end of the endoscope closer to the operator is called the “proximal end”, and the end farther from the operator is called the “distal end”, and the “proximal end” and “distal end” of any component of the endoscope are defined based on this principle.

In the related art, the joystick mechanism often utilizes a ball head structure to achieve movable connections with the operating handle. However, frequent manipulation of the joystick during operation can lead to loosening of the ball head joint. This not only compromises operational tactile feedback but also adversely affects precision in curvature adjustment.

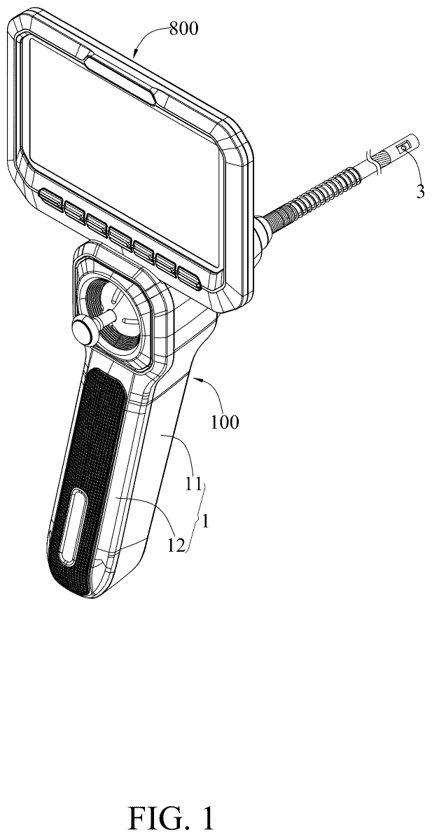

As illustrated in FIG. 1, according to some embodiments, an endoscope includes an operating handle 100 and a display screen 800 connected to the operating handle 100. The operating handle 100 includes a lens assembly 3. During the inspection process of an engineering operation, the lens assembly 3 is configured to shoot the area that cannot be reached by sight, and the shot image is displayed on the display screen 800.

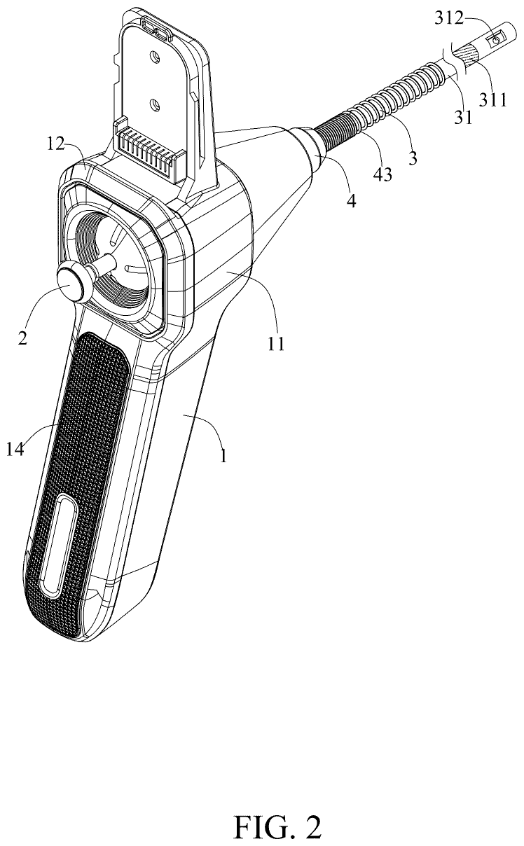

As illustrated in FIG. 2, in some embodiments, the operating handle 100 also includes a housing 1, a joystick mechanism 2, and a protective coupling assembly 4.

In some embodiments, a first portion of the joystick mechanism 2 is disposed in the housing 1 and is fixedly connected to an inner sidewall of the housing 1. A second portion of the joystick mechanism 2 extends out of the housing 1, allowing a user to operate. The lens assembly 3 is connected to the housing 1. A proximal end of the lens assembly 3 extends into the housing 1 and is connected to the joystick mechanism 2. A distal end of the lens assembly 3 extends to a side away from the housing 1. The protective coupling assembly 4 is sleeved on the proximal end of the lens assembly 3 and connected to the housing 1. The proximal end of the lens assembly 3 is fixedly connected to the housing 1 by the protective coupling assembly 4.

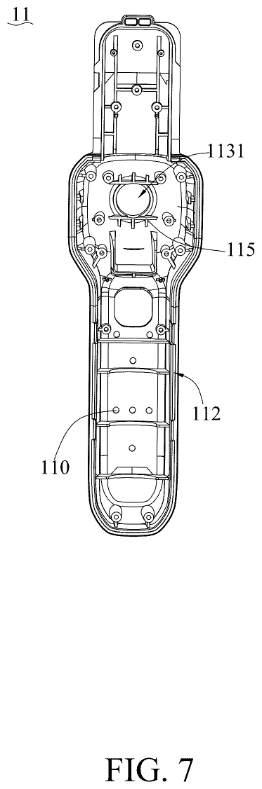

As illustrated in FIG. 2 to FIG. 8, in some embodiments, the housing 1 includes a first housing 11 and a second housing 12 detachably assembled with the first housing 11. In some embodiments, a tab 111 is protruded on an end surface of the first housing 11 proximal to the second housing 12, and a snap-fit block 121 is protruded on an inner sidewall of the second housing 12. The first housing 11 and the second housing 12 are detachably connected by the mutual engagement of the tab 111 and the snap-fit block 121.

In some embodiments, the end surface of the first housing 11 proximal to the second housing 12 is defined with a groove 112. A raised strip 122 is provided on an end surface of the second housing 12 proximal to the first housing 11. When the first housing 11 and the second housing 12 are assembled, the raised strip 122 is inserted into the groove 112, thereby improving the tightness of the assembly between the first housing 11 and the second housing 12.

In some embodiments, the detachable connection between the tab 111 and the snap-fit block 121 allows the first housing 11 and the second housing 12 in a pre-installed state. In this manner, the first housing 11 and the second housing 12 are further fixedly assembled by fastening screws, thereby improving the tightness of the assembly between the first housing 11 and the second housing 12. In some embodiments, the first housing 11 and the second housing 12 are detachably assembled only by the tab 111 and the snap-fit block 121, or detachably assembled only by fastening screws.

In some embodiments, the first housing 11 also includes a first mounting portion 113 arranged on the end surface of the first housing 11 away from the second housing 12. The first mounting portion 113 extends to the distal end to form a protruding structure.

In some embodiments, the first mounting portion 113 is substantially truncated, and its cross section gradually increases from the distal end to the proximal end. A distal end surface of the first mounting portion 113 is defined with a first mounting hole 1131 extending from the first housing 11 along its longitudinal direction. A proximal portion of the lens assembly 3 extends into the first mounting hole 1131 and is connected to the first mounting portion 113 by the protective coupling assembly 4. Such a configuration not only facilitates the protective coupling assembly 4 to connect the lens assembly 3 to the first mounting portion 113, but also enables the lens assembly 3 to be smoothly connected to the first housing 11, thereby improving the integrity of the operating handle 100.

As illustrated in FIG. 2 to FIG. 8, in some embodiments, a first decorative piece 13 is provided on the first housing 11. In some embodiments, the first housing 11 is defined with a first insertion hole 110, and one side proximal to the first housing 11 of the first decorative piece 13 is protruded with a first insertion column 131. The first insertion column 131 is inserted into the first insertion hole 110 to allow the first decorative piece 13 to be detachably assembled with the first housing 11. In some other embodiments, the first decorative piece 13 is detachably engaged with the first housing 11 by snap-fit fixation, adhesive fixation, bolt fixation, etc.

In some embodiments, one side of the first decorative piece 13 away from the first housing 11 is defined with a finger groove 130. The finger groove 130 is provided in multiple numbers and arranged linearly. When holding the operating handle 100, a user can place a finger in the finger groove 130, improving the grip of the operating handle 100 and creating an anti-slip effect. In some embodiments, four finger grooves 130 are provided, and in some other embodiments, three, two, etc. finger grooves 130 are provided.

As illustrated in FIG. 2 to FIG. 8, in some embodiments, a second decorative piece 14 is provided on the second housing 12. In some embodiments, the second housing 12 is defined with a second insertion hole 120, and a second insertion column 141 is protruded on one side of the second decorative piece 14 proximal to the second housing 12. The second insertion column 141 is inserted into the second insertion hole 120, achieving detachable assembly between the second decorative piece 14 with the second housing 12. In some other embodiments, the second decorative piece 14 is detachably assembled with the second housing 12 by snap-fit fixation, adhesive fixation, bolt fixation, etc. In some embodiments, a plurality of tips is provided on one side of the second decorative piece 14 away from the second housing 12, enhancing the anti-slip effect of the second decorative piece 14.

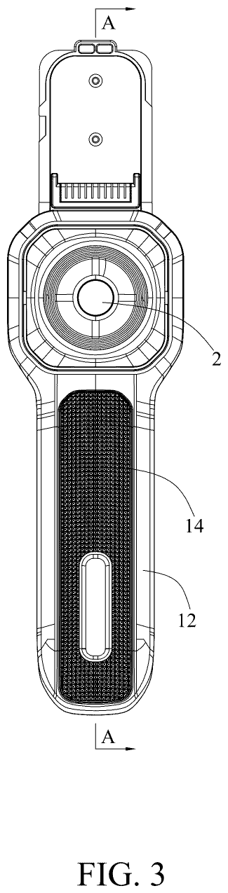

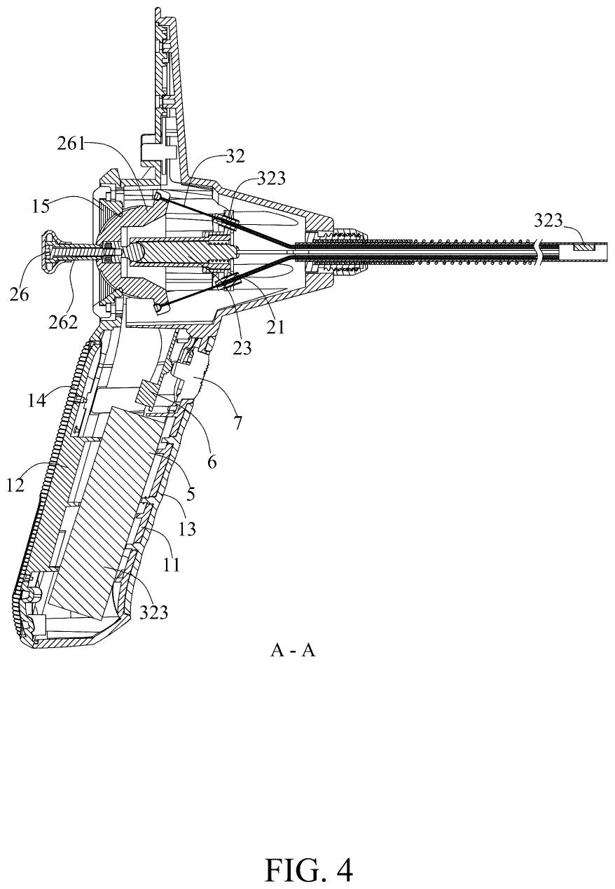

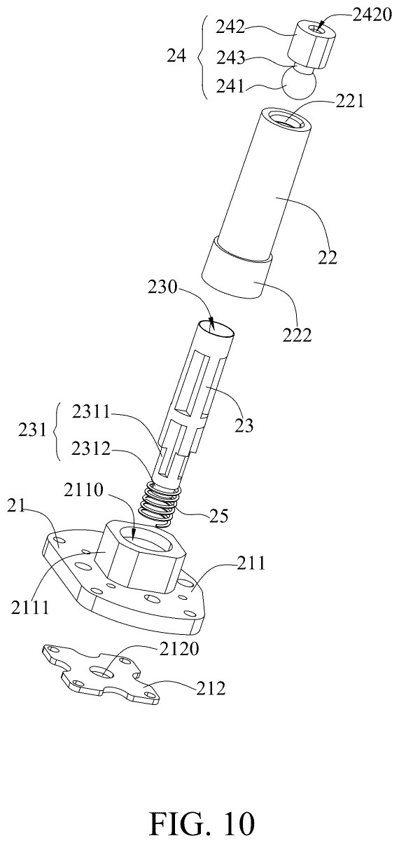

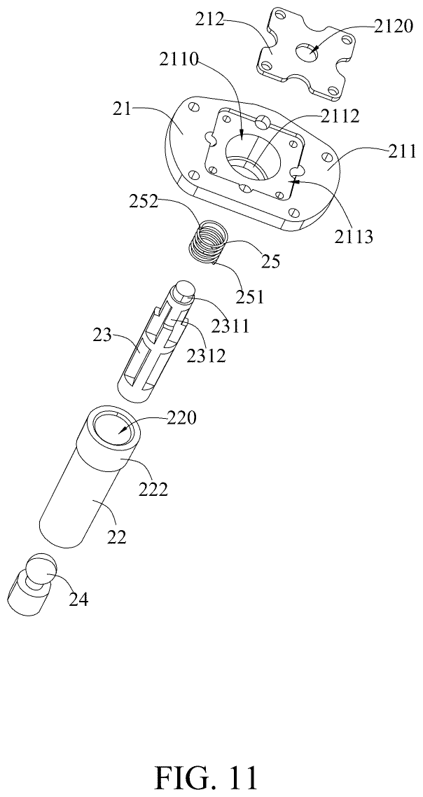

As illustrated in FIG. 3, FIG. 4 and FIG. 9 to FIG. 11, in some embodiments, the joystick mechanism 2 includes a mounting seat 21, an outer sleeve 22, a support column 23, a head joint 24, a first elastic member 25 and a joystick assembly 26.

In some embodiments, the mounting seat 21 is fixedly mounted on an inner wall of the first housing 11 by fastening screws. The outer sleeve 22 is connected to the mounting seat 21, and an end of the outer sleeve 22 away from the mounting seat 21 is provided with a first retention portion 221. The support column 23 is disposed in a cavity 220 of the outer sleeve 22. The support column 23 can move along an axial direction of the cavity 220. An end of the support column 23 away from the mounting seat 21 is defined with a first mounting groove 230. The head joint 24 has a ball head 241, which is disposed in the first mounting groove 230 and is restricted between the support column 23 and the first retention portion 221. The ball head 241 can move in the first mounting groove 230, and one end of the head joint 24 away from the ball head 241 extends out of the cavity 220 of the outer sleeve 22 and is connected to the joystick assembly 26. The first elastic member 25 is arranged in the cavity 220 of the outer sleeve 22. The first elastic member 25 has a first end 251 and a second end 252, the first end 251 is connected to the support column 23, and the second end 252 is connected to the mounting seat 21. The first elastic member 25 can squeeze the support column 23 to a side away from the mounting seat 21, allowing a groove wall of the first mounting groove 230 to abut against the ball head 241. The joystick assembly 26 is connected to a portion of the head joint 24 that extends out of the outer sleeve 22. The joystick assembly 26 is configured to control the head joint 24, allowing the head joint 24 to move in the first mounting groove 230.

In some embodiments, after the joystick mechanism 2 is assembled, the first elastic member 25 is in a compressed state, squeezing the support column 23 to the side away from the mounting seat 21 with its restoring force. When in operation, the groove wall of the first mounting groove 230 is always fitted with the ball head 241, increasing the friction force of the ball head 241 relative to the first mounting groove 230, and creating a damping force to the head joint 24. The joystick mechanism 2 configured in this way, on the one hand, improves the operating experience of the joystick mechanism; on the other hand, the setting of the first elastic member 25 enables the support column 23 to squeeze the ball head 241 against the first retention portion 221, allowing the ball head 241 to be stably and firmly confined between the support column 23 and the first retention portion 221. This reduces the occurrences of loosening of the ball head 241 due to frequent operation, thereby improving the accuracy of the operation of this industrial endoscope.

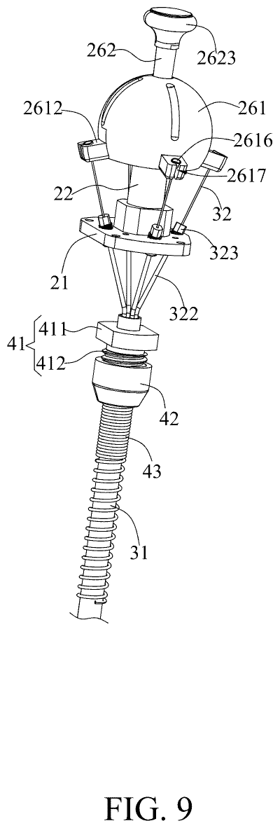

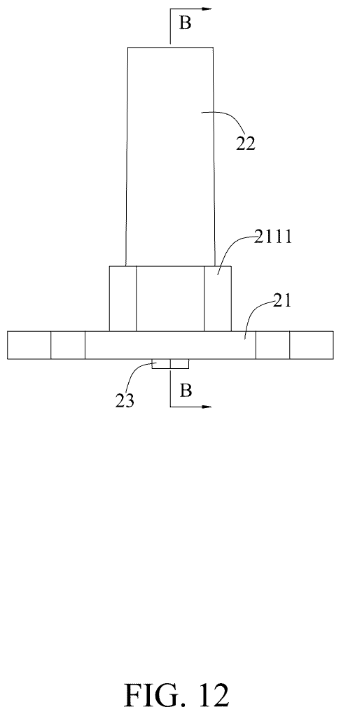

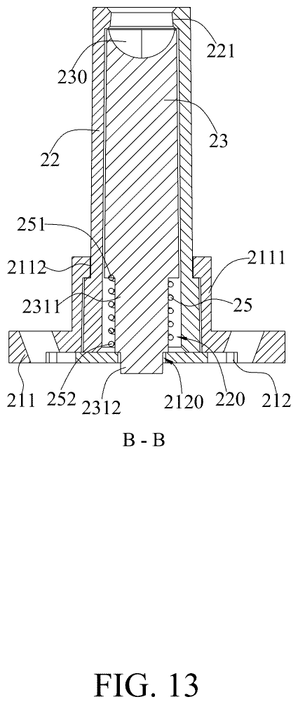

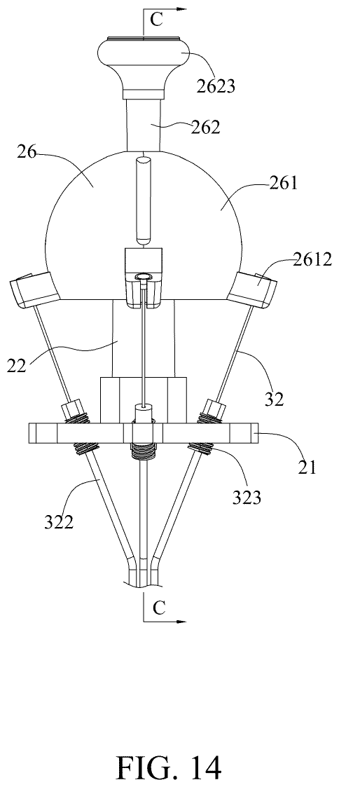

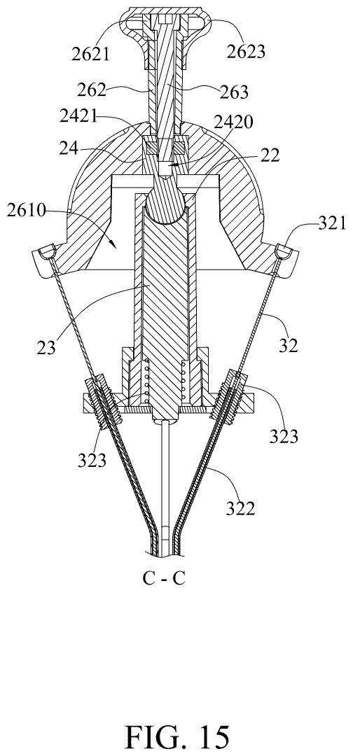

As illustrated in FIG. 9 to FIG. 15, in some embodiments, the mounting seat 21 includes a seat body 211 and a bottom plate 212. The seat body 211 has a mounting boss 2111, and the seat body 211 is defined with an assembly groove 2110 extending into the mounting boss 2111. A portion of the outer sleeve 22 is disposed in the assembly groove 2110. The bottom plate 212 is arranged on a side of the seat body 211 away from the mounting boss 2111. The bottom plate 212 is detachably connected to the seat body 211, allowing the outer sleeve 22 to be confined in the assembly groove 2110.

In some embodiments, an end of the mounting boss 2111 away from the bottom plate 212 is provided with a second retention portion 2112, and the second retention portion 2112 protrudes out of an inner sidewall of the assembly groove 2110. An end, proximal to the seat body 211, of the outer sleeve 22 is provided with an enlarged portion 222, and the enlarged portion 222 is secured in the assembly groove 2110 by the second retention portion 2112 and the bottom plate 212.

In some embodiments, the end surface of the seat body 211 is defined with a sinking groove 2113 for mounting the bottom plate 212, and the sinking groove 2113 is in communication with the assembly groove 2110. The bottom plate 212 is accommodated in the sinking groove 2113 and is fixedly assembled with the seat body 211 by fastening screws, thereby securing the enlarged portion 222.

In some embodiments, an end, proximal to the mounting seat 21, of the support column 23 is provided with a reduced portion 231, and the first elastic member 25 is sleeved on the reduced portion 231. When the first elastic member 25 undergoes elastic deformation, the reduced portion 231 provides guide for the first elastic member 25 during the deformation process, creating a more stable deformation process for the first elastic member 25.

In some embodiments, the bottom plate 212 is defined with a through hole 2120. The reduced portion 231 includes a first portion 2311 and a second portion 2312 connected to one end, proximal to the seat body 211, of the first portion 2311. The second portion 2312 is defined with the through hole 2120, and a radial dimension of the first portion 2311 is greater than a radial dimension of the through hole 2120. A step surface is formed at a position where the first portion 2311 and the second portion 2312 are connected, and the step surface abuts against the bottom plate 212 to prevent the support column 23 from slipping out of the cavity 220 of the outer sleeve 22 from the through hole 2120.

As illustrated in FIG. 9 to FIG. 15, in some embodiments, the head joint 24 also includes an assembly portion 242 and a connecting portion 243. The assembly portion 242 and the connecting portion 243 are both located outside the outer sleeve 22, and the assembly portion 242 is connected to the ball head 241 by the connecting portion 243. An end surface, away from the ball head 241, of the assembly portion 242 is defined with an assembly hole 2420. A flaring structure is provided at an opening of the assembly hole 2420, facilitating the joystick assembly 26 to be inserted into the assembly hole 2420 and connected with the assembly portion 242. In some embodiments, a fastening sleeve 2421 is embedded in the assembly portion 242, and the fastening sleeve 2421 is configured to fit with the portion of the joystick assembly 26 extending into the assembly hole 2420, to achieve detachable assembly of the head joint 24 and the joystick assembly 26.

In some embodiments, the assembly portion 242 is cylindrical, and an outer sidewall of the assembly portion 242 is defined with a flat surface extending along the axial direction. When the assembly portion 242 is assembled with the joystick assembly 26, the flat surface prevents the assembly portion 242 from rotating around its own axis relative to the joystick assembly 26, thereby improving the control performance of the joystick assembly 26 on the head joint 24.

In some embodiments, a radial dimension of the cross section of the assembly portion 242 is greater than a radial dimension of the connecting portion 243, improving the structural strength of the assembly portion 242 and enhancing the stability of the assembly of the head joint 24 and the joystick assembly 26.

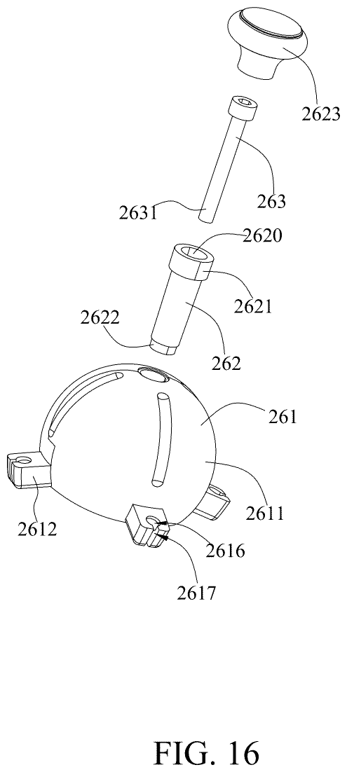

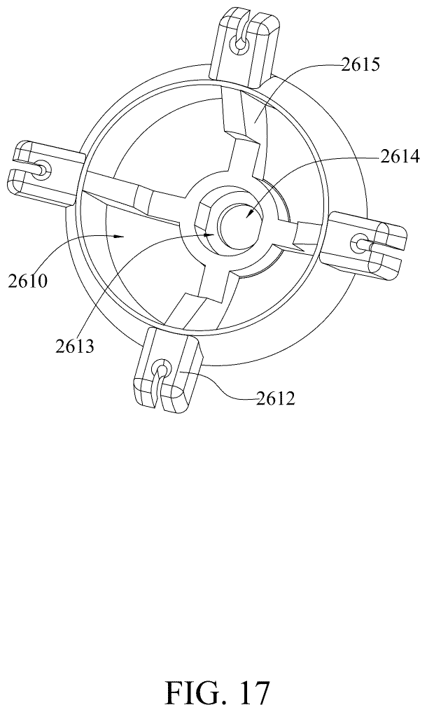

As illustrated in FIG. 9 to FIG. 17, in some embodiments, the joystick assembly 26 includes a linkage member 261, an operating rod 262, and a fastener 263. The linkage member 261 is connected to a portion, extending out of the outer sleeve 22, of the head joint 24. The operating rod 262 has an operating end 2621 and an assembly end 2622. The operating end 2621 extends out of the second housing 12, facilitating the operation of the joystick mechanism 2. The assembly end 2622 extends into the linkage member 261. The operating rod 262 has a channel 2620 that axially extends from the operating end 2621 to the assembly end 2622. The fastener 263 has a connecting end 2631. During assembly, the fastener 263 extends through the channel 2620, and the connecting end 2631 extends through the channel 2620 and is connected to the head joint 24, locking and securing the operating rod 262 and the linkage member 261. Therefore, the head joint 24, the linkage member 261 and the operating rod 262 can move synchronously, and the head joint 24 and the linkage member 261 can be controlled by the operating rod 262.

In some embodiments, the linkage member 261 includes a linkage body 2611 and a linkage end 2612 arranged on an outer sidewall of the linkage body 2611. The linkage body 2611 has a receiving cavity 2610. One side, away from the mounting seat 21, of the outer sleeve 22 is accommodated in the receiving cavity 2610. A bottom of the receiving cavity 2610 is defined with a receiving groove 2613, and the assembly portion 242 is accommodated in the receiving groove 2613. A bottom of the receiving groove 2613 is defined with a connecting hole 2614 that extending through the linkage body 2611, and the assembly end 2622 of the operating rod 262 is inserted into the connecting hole 2614. The connecting end 2631 of the fastener 263 is inserted into the assembly hole 2420 and fits with the fastening sleeve 2421. There are multiple linkage ends 2612, and the multiple linkage ends 2612 are arranged on the outer wall of the linkage body 2611 in an annular manner with equal spacing. The linkage ends 2612 are configured to connect with the lens assembly 3, thus controlling the bending direction and bending degree of the distal end of the lens assembly 3.

In some embodiments, the linkage body 2611 is hemispherical, and a reinforcing rib 2615 is provided on an inner sidewall of the receiving cavity 2610. The reinforcing rib 2615 is arranged corresponding to a position of the linkage end 2612, improving the structural strength of the linkage end 2612 and the stability of the operation process.

In some embodiments, an operating sleeve 2623 is provided on the operating end 2621 of the operating rod 262 to enhance the comfort of holding the operating end 2621, thereby improving operation experience. In some embodiments, the operating sleeve 2623 is a rubber sleeve. In some other embodiments, the operating sleeve 2623 is a plastic sleeve or a metal sleeve.

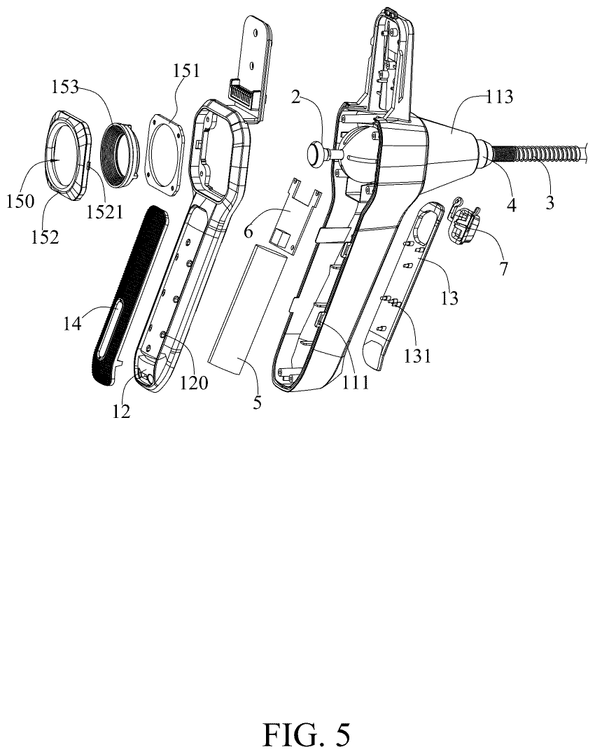

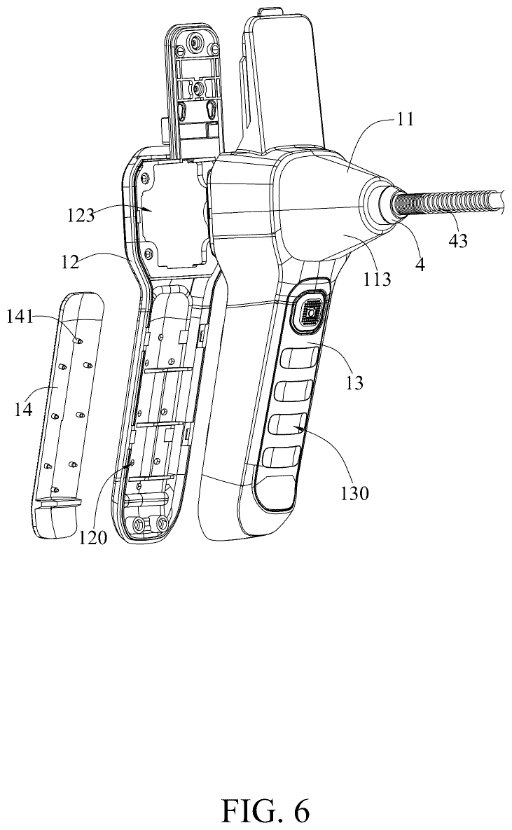

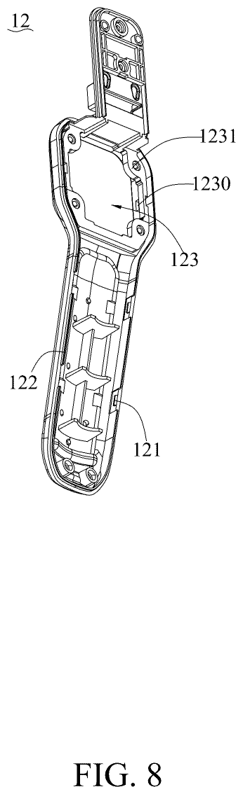

As illustrated in FIG. 2, FIG. 5, FIG. 6 and FIG. 8, in some embodiments, a mounting port 123 is provided on the second housing 12. The operating end 2621 extends to a side away from the first housing 11 to extend out of the mounting port 123 for operation. The housing 1 also includes a clamping assembly 15. A plurality of mounting platforms 1231 are protruded from a sidewall of the mounting port 123, and the clamping assembly 15 is mounted on the mounting platform 1231. The clamping assembly 15 is provided with an operating port 150 for the operating rod 262 to pass through, and the operating rod 262 can move in the operating port 150 to control the lens assembly 3 by the joystick assembly 26.

In some embodiments, the clamping assembly 15 includes a fixing plate 151, a cover plate 152 and a clamping plate 153. The fixing plate 151 is fixedly mounted on the mounting platform 1211 by fastening screws, and the cover plate 152 is arranged on one side of the fixing plate 151 away from the first housing 11. The cover plate 152 is detachably assembled with the second housing 12 to clamp the clamping plate 153 on the fixing plate 151. An end, away from the cover plate 152, of the clamping plate 153 is abutted against an outer wall of the linkage body 2611 to secure the linkage body 2611, thereby improving the stability of the assembly of the linkage body 2611 and facilitating the operation of the joystick assembly 26.

In some embodiments, a snap member 1230 is provided on the sidewall of the mounting port 123. A block 1521 is protruded on a sidewall of the cover plate 152. The cover plate 152 is connected to the second housing 12 by the block 1521 and the snap member 1230.

As illustrated in FIG. 2 to FIG. 4, in some embodiments, the lens assembly 3 includes a lens tube 31 and a traction line 32 extending through the lens tube 31. A proximal end of the lens tube 31 is connected to the housing 1. A distal end of the traction line 32 is connected to a distal end of the lens tube 31, and a proximal end of the traction line 32 extends through a proximal end surface of the lens tube 31 to be connected to the linkage member 261. The operating rod 262 is configured to control the head joint 24 to move relative to the support column 23, drive the linkage member 261 to move, and pull the traction line 32, thereby controlling the bending of the distal end of the lens tube 31.

In some embodiments, the distal end of the lens tube 31 is provided with a bending section 311 and a camera 312. The distal end of the traction line 32 is fixedly connected to the bending section 311. The camera 312 is embedded in the bending section 311, and the camera 312 can move synchronously with the bending section 311 to adjust the shooting angle.

As illustrated in FIG. 9 to FIG. 17, in some embodiments, the linkage end 2612 is defined with a slot 2616, and a sidewall of the slot 2616 is defined with a mounting opening 2617. The proximal end of the traction line 32 is connected to a fixing block 321, and the traction line 32 enters the slot 2616 from the mounting opening 2617, and is secured within the slot 2616 by the fixing block 321. When the operating rod 262 is operated, the traction line 32 is pulled by the linkage member 261 to adjust the bending direction and bending angle of the bending section 311.

In some embodiments, four linkage ends 2612 are provided, and the four linkage ends 2612 are distributed in an annular manner at equal intervals on the outer wall of the linkage body 2611. In other words, in the direction from the proximal end to the distal end, the four linkage ends 2612 are respectively located at the upper, lower, left, and right positions of the linkage body 2611, thereby adjusting the bending section 311 in four directions. In some other embodiments, two linkage ends 2612 are provided, thereby realizing bidirectional adjustment of the bending section 311.

In some embodiments, the seat body 211 is defined with a threaded hole, and the traction line 32 extends through the threaded hole to be connected to the linkage end 2612. A traction guide tube 322 is sleeved outside the traction line 32, and the traction guide tube 322 also extends through a lens tube 31. A proximal end of the traction guide tube 322 is fixedly connected with a fixing member 323. The fixing member 323 is configured to assemble with the threaded hole to secure the proximal end of the traction guide tube 322. The distal end of the traction guide tube 322 is fixedly connected to the bending section 311. The fixing member 323 is defined with a perforation along the axial direction of the threaded hole, allowing the traction line 32 to pass through. An outer wall of the fixing member 323 is provided with an external thread, and the fixing member 323 is threadedly fixedly connected to the seat body 211 by the external thread and the threaded hole.

In some embodiments, the setting of the traction guide tube 322 can effectively reduce the occurrences of the traction line 32 being broken due to friction with the inner wall of the seat body 211 or the lens tube 31 during the pulling process, thereby improving the service life of the lens assembly 3.

In some embodiments, a power supply 5, a control circuit board 6 and a control button 7 are also provided in the housing 1. In some embodiments, the control circuit board 6 is electrically connected to the power supply 5 by a wire, and the camera 312 is electrically connected to the control circuit board 6 by a wire. The first decorative member 13 and the first housing 11 each is provided with a hole for installing the control button 7, and the control button 7 is disposed on the control circuit board 6, and the first decorative member 13 protrudes from a perforation of the operation end, allowing the user to operate.

In some embodiments, the protective coupling assembly 4 includes a connecting member 41, a locking member 42 and a winding member 43. The connecting member 41 is arranged in the housing 1 and sleeved on the proximal end of the lens tube 31. The connecting member 41 has a connecting section 411 and an assembly section 412. The connecting section 411 fits with the inner sidewall of the housing 1, so as to be confined in the housing 1. One end of the assembly section 412 is connected to the connecting section 411, and another end of the assembly section 412 extends out of the housing 1 to engage with the locking member 42. The locking member 42 is sleeved on the proximal end of the lens tube 31 and assembled with the assembly section 412 of the connecting member 41, allowing the locking member 42 and the connecting section 411 to clamp the sidewall of the housing 1, further achieving the assembly of the protective coupling assembly 4 and the housing 1. The winding member 43 is spirally wound on an outer sidewall of the proximal end of the lens tube 31. The proximal end of the winding member 43 is fixedly connected to the connecting member 41, and the winding member 43 spirally wraps around the lens tube 31 from the proximal end and extends to the distal end.

In some embodiments, an external thread is provided on an outer wall of the assembly section 412, the locking member 42 is defined with a threaded hole. The connecting member 41 and the locking member 42 are threaded to clamp the first housing 11. A strip 115 is provided on the inner wall of the first housing 11, and the strip 115 is located near an opening of the first mounting hole 1131. When the connecting section 411 is assembled, the connecting section 411 cooperates with the strip 115 to prevent the connecting member 41 from rotating relative to the first housing 11, allowing the locking member 42 and the connecting member 41 to clamp the first housing.

In some embodiments, the connecting member 41 and the locking member 42 are threaded together to clamp the first housing 11, thereby the operating handle can be replaced with different lens assemblies 3 due to the detachable connection between the connecting member 41 and the locking member 42.

As illustrated in FIG. 2 to FIG. 4 and FIG. 9, in some embodiments, the connecting member 41 is defined with a circular hole, and the lens tube 31 extends through the circular hole, and the proximal end of the lens tube 31 extends out of the connecting section 411. A proximal end of the winding member 43 is arranged between the outer wall of the lens tube 31 and the inner wall of the circular hole. The winding member 43 is spirally wound to form a spring-shaped protective structure. The proximal end of the winding member 43 is densely wound and the distal end of the winding member 43 is sparsely wound. The winding member 43 arranged in this way has high structural strength at its proximal end and good flexibility at its distal end, preventing the proximal end of the lens tube 31 from bending without affecting the overall flexibility of the lens tube 31.

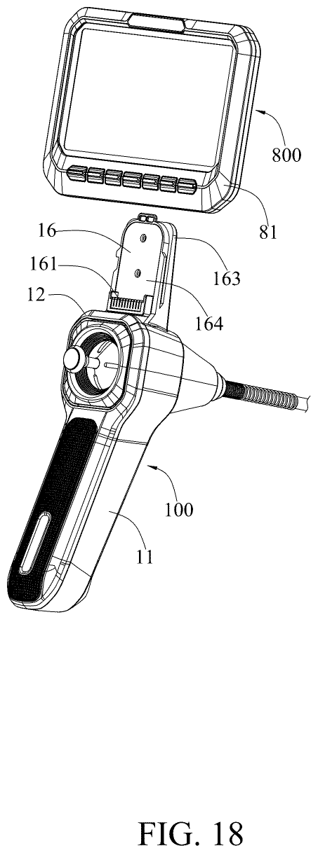

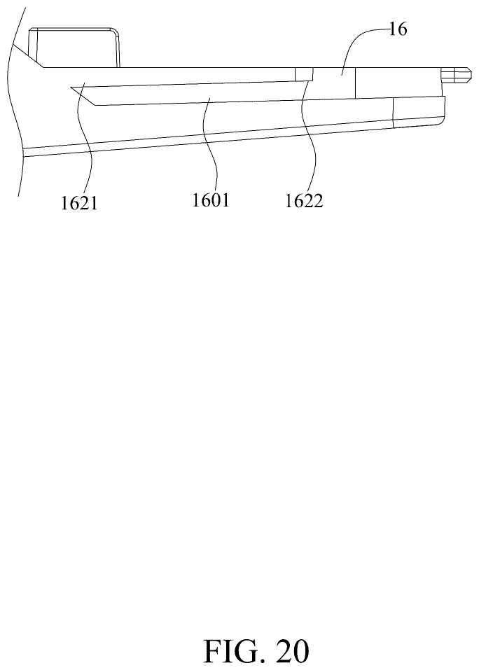

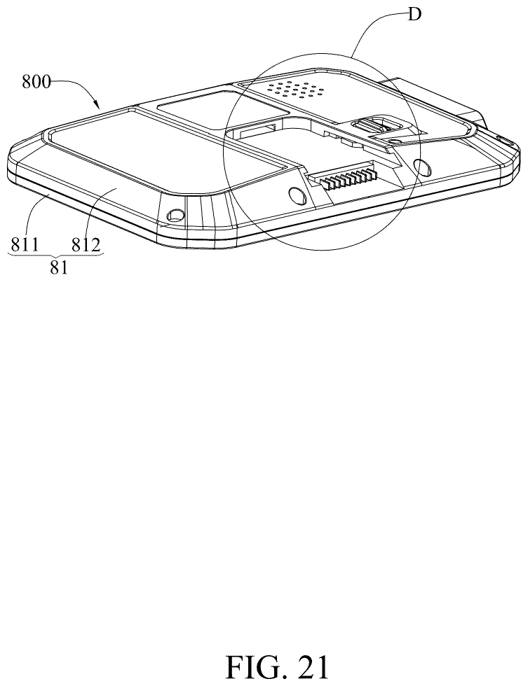

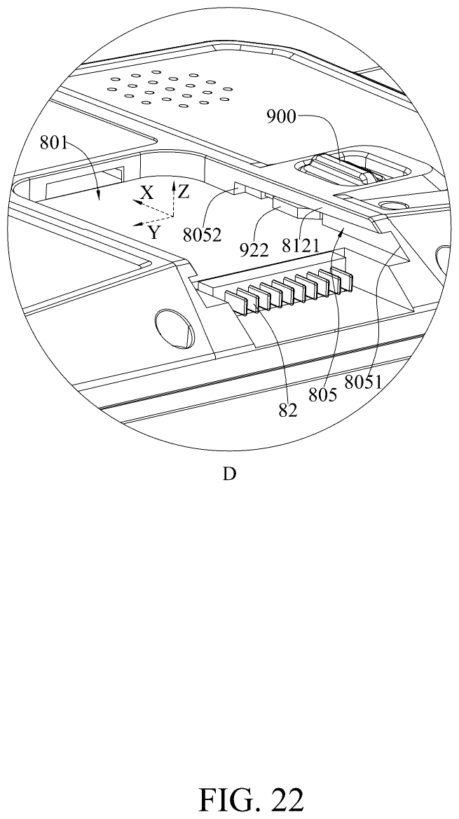

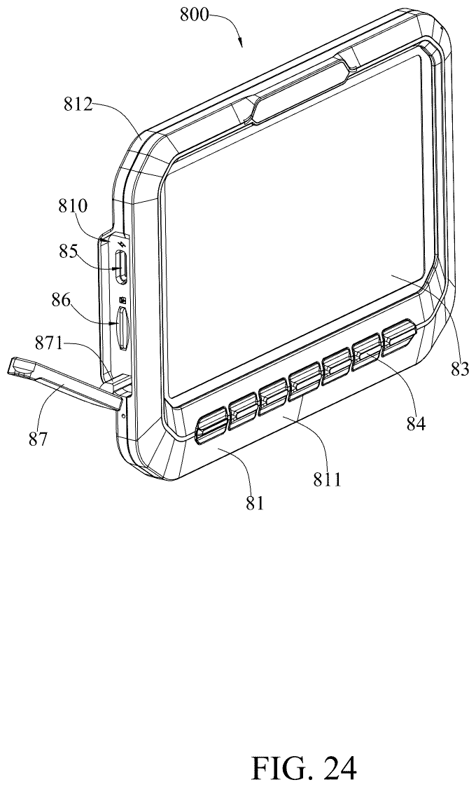

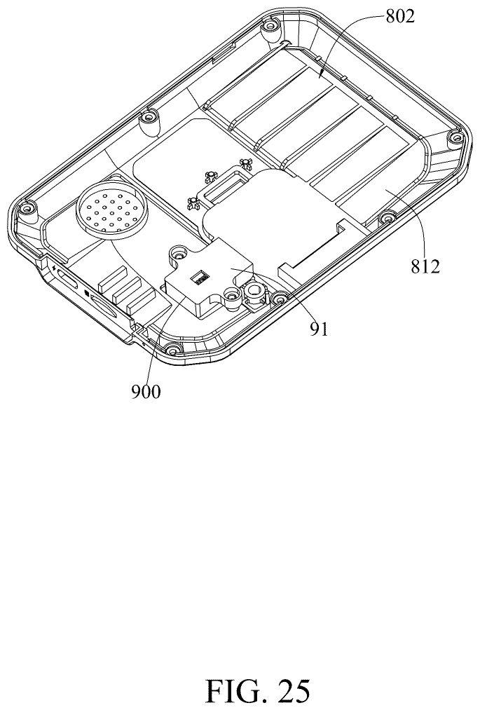

As illustrated in FIG. 1, FIG. 18 to FIG. 26, in some embodiments, a connector 16 is extending from the outer wall of the operating handle 100, and a first connecting terminal 161 is provided on the connector 16. The display screen 800 is detachably connected to the operating handle 100. When necessary, the display screen 800 is separated from the operating handle 100, thereby reducing its dimension, which is convenient for packaging, transportation or carrying. The display screen 800 includes a casing 81 and a second connecting terminal 82. The casing 81 is defined with a connecting groove 801, and the connector 16 is inserted into the connecting groove 801 to realize the detachable assembly between the operating handle 100 and the display screen 800. A fixed end of the second connecting terminal 82 is assembled in an inner cavity 802 of the casing 81, and an insertion end of the second connecting terminal 82 extends into the connecting groove 801. The second connecting terminal 82 is inserted into the first connecting terminal 161, allowing a conductive circuit formed between the operating handle 100 and the display screen 800.

In some embodiments, an inner sidewall of the connecting groove 801 is defined with a snap-fit channel 803 communicating with the inner cavity 802 of the casing 81. The endoscope further includes a snap-fit assembly 900, which includes a first portion and a second portion. The first portion of the snap-fit assembly 900 is disposed in the inner cavity 802 of the casing 81 and is fixedly connected to the inner sidewall of the casing 81. The second portion of the snap-fit assembly 900 is accommodated in the snap-fit channel 803 and is connected to the first portion of the snap-fit assembly 900. The second portion of the snap-fit assembly 900 can move relative to the snap-fit channel 803, so as to extend out or retract into the snap-fit channel 803. The second portion of the snap-fit assembly 900 is configured to engage or disengage with the connector 16 to achieve locking or unlocking of the connector 16. In some embodiments, the setting of the snap-fit assembly 900 enables the display screen 800 and the operating handle 100 to be quickly disassembled and assembled, which not only improves the stability of the connection between the display screen 800 and the operating handle 100, but also facilitates the disassembly, use, transportation or carrying of the endoscope.

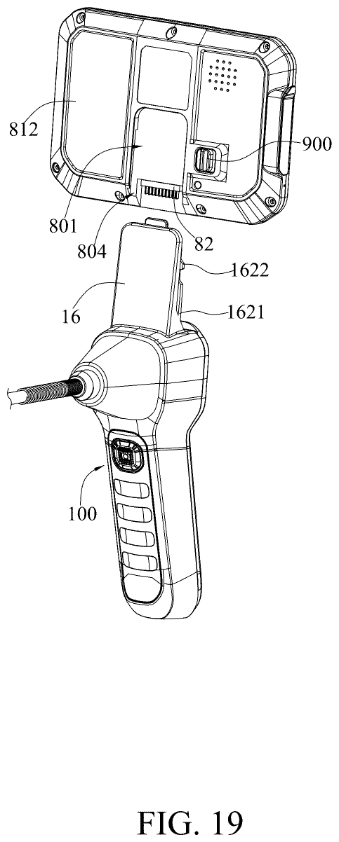

As illustrated in FIG. 19 to FIG. 26, in some embodiments, the casing 81 includes a front casing 811 and a rear cover 812 fixedly assembled with the front casing 811. An outer sidewall of the rear cover 812 is defined with the connecting groove 801, and the connecting groove 801 has an opening 804 for insertion of the connector 16, allowing the connector 16 to enter the connecting groove 801 from the opening 804 and be detachably connected to the rear cover 812.

In some embodiments, a first engaging groove 805 extending along a first direction X is defined by the sidewall of the connecting groove 801, and a first end 8051 of the first engaging groove 805 is in communication with the opening 804. A third retention member 162 is provided on a sidewall of the connector 16, and the third retention member 162 extends into the first engaging groove 805 from the first end 8051 of the first engaging groove 805. The third retention member 162 is configured to insert into the first engaging groove 805, securing the connector 16 in the connecting groove 801 in a second direction Z, and improving the stability of the connector 16 and the connecting groove 801.

In some embodiments, the third retention member 162 is inserted into the first engaging groove 805 and locked by the snap-fit assembly 900 to achieve detachable assembly between the display screen 800 and the operating handle 100. The detachable assembly between the display screen 800 and the operating handle 100 can also be achieved by inserting the third retention member 162 into the first engaging groove 805. In this manner, the third retention member 162 and the first engaging groove 805 have an interference fit.

In some embodiments, the third retention member 162 has a bottom end 1621 and a head end 1622, the bottom end 1621 is an end proximal to the operating handle 100, and the head end 1622 is an end away from the operating handle 100. The first end 8051 of the first engaging groove 805 is an end proximal to the bottom end of the connector 16 after the connector 16 is assembled with the connecting groove 801. The second end 8052 of the first engaging groove 805 is an end proximal to the head end 1622 after the connector 16 is assembled with the connecting groove 801. When the third retention member 162 is assembled with the first engaging groove 805, the head end 1622 of the third retention member 162 is first engaged with the first end 8051 of the first engaging groove 805, and then the third retention member 162 is further pushed toward the second end 8502 of the first engaging groove 805, allowing the head end 1622 of the third retention member 162 to abut against an inner wall of the second end 8052 of the first engaging groove 805.

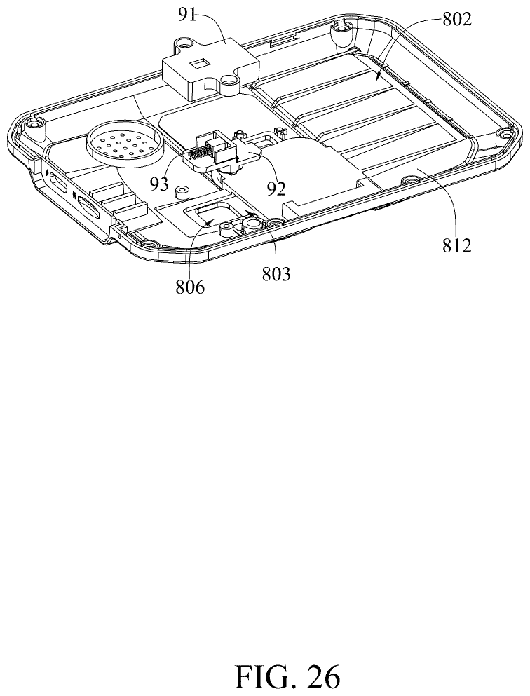

As illustrated in FIG. 19 and FIG. 26, in some embodiments, a cross-sectional dimension of bottom end 1621 is greater than a cross-sectional dimension of the head end 1622. Correspondingly, a dimension of the first end 8051 of the first engaging groove 805 is greater than a dimension of the second end 8052. Such a configuration makes it easier for the head end 1622 of the third retention member 162 to be inserted from the first end 8051 of the first engaging groove 805, improving the convenience of assembling the third retention member 162 with the first engaging groove 805. In addition, the cross-sectional dimension of the bottom end 1621 of the third retention member 162 is greater than the cross-sectional dimension of the head end 1622, enabling the third retention member 162 to be wedge-shaped. The wedge-shaped structure makes it easy for the engagement of the first engaging groove 805 and the third retention member 162, enhancing tightness between the first engaging groove 805 and the third retention member 162 after being engaged together, thus improving the stability of the assembly of the connector 16 with the connecting groove 801.

In some embodiments, a fourth retention portion 8121 is formed on the sidewall of the connecting groove 801. The sidewall of the connector 16 is defined with a second engaging groove 1601. When the connecting groove 801 is engaged with the connector 16, the fourth retention portion 8121 is engaged with the second engaging groove 1601, and the connector 16 is further restricted in the second direction Z, thereby further improving the stability of the connection between the connector 16 and the connecting groove 801.

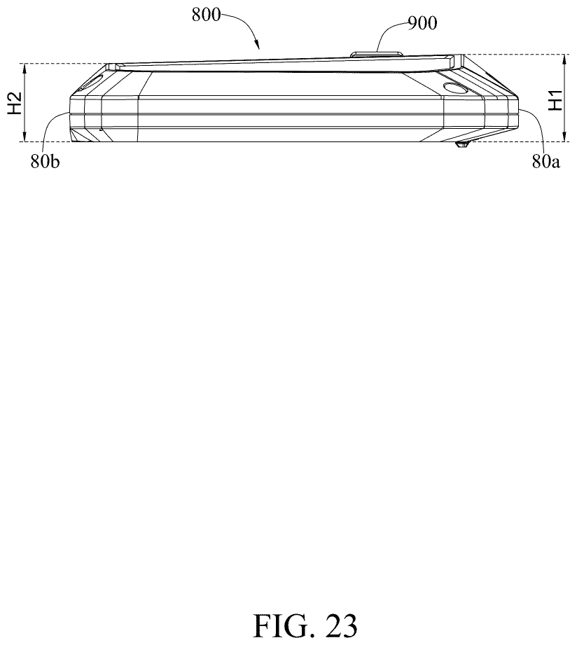

As illustrated in FIG. 19 and FIG. 23, in some embodiments, one side, connected to the connector 16, of the display screen 800 is an assembly side 80a. Another side, opposite to the assembly side 80a, of the display screen 800 is a non-assembly side 80b. A thickness H1 of the assembly side 80a is greater than a thickness H2 of the non-assembly side 80b. As such, when the display screen 800 is assembled with the operating handle 100, the center of gravity of the display screen 800 is closer to the side where the operating handle 100 is located, thereby further improving the stability of the connection between the display screen 800 and the operating handle 100.

As illustrated in FIG. 8 to FIG. 10, in some embodiments, the display screen 800 further includes a screen 83, a circuit board, a control button 84, a connection port 85, a memory expansion port 86, and a dustproof seal 87.

In some embodiments, a viewing window is provided on the front casing 811. The screen 83 is arranged in the inner cavity 802, and a display end surface corresponds to the viewing window, allowing the display content of the screen 83 accessible via the viewing window. The circuit board is connected to the second connection terminal 82, the control button 84, the connection port 85, and the memory expansion port 86. The circuit board is configured to perform data transmission, signal transmission, and circuit conduction. There are multiple control buttons 84, and they are all arranged on the front casing 811. The front casing 811 is also provided with an installation port 810 in communication with the inner cavity 802, and the connection port 85 and the memory expansion port 86 are located in the installation port 810. The connection port 85 is a Type-C interface; the memory expansion port 86 is an SD card insertion port or a TF card insertion port. The dustproof seal 87 has an anti-slip foot 871. The anti-slip foot 871 is connected to the front casing 811 to prevent the dust seal 87 from detaching from the front casing 811 after being opened, facilitating closing the installation port 810 after using the connection port 85 and the memory expansion port 86. The anti-slip foot 871 has rigidness, increasing the opening angle of the dust seal 87 and facilitating the use of the connection port 85 and the memory expansion port 86.

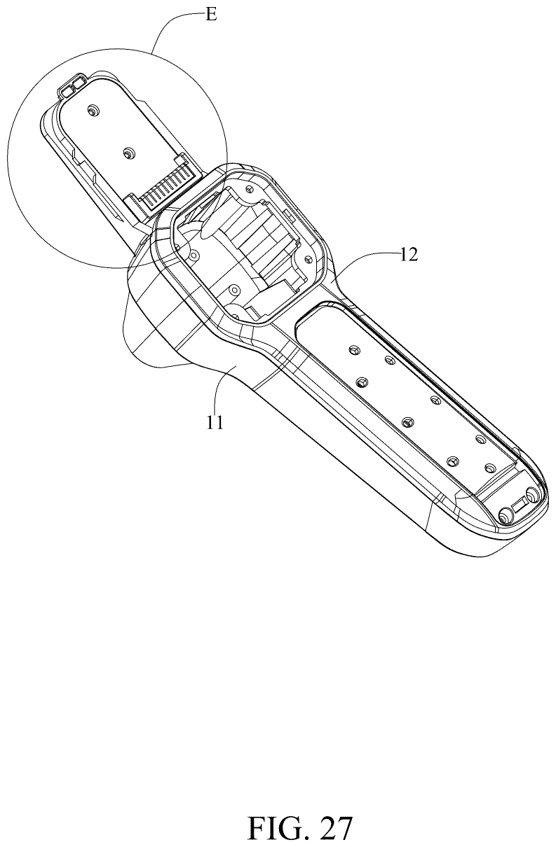

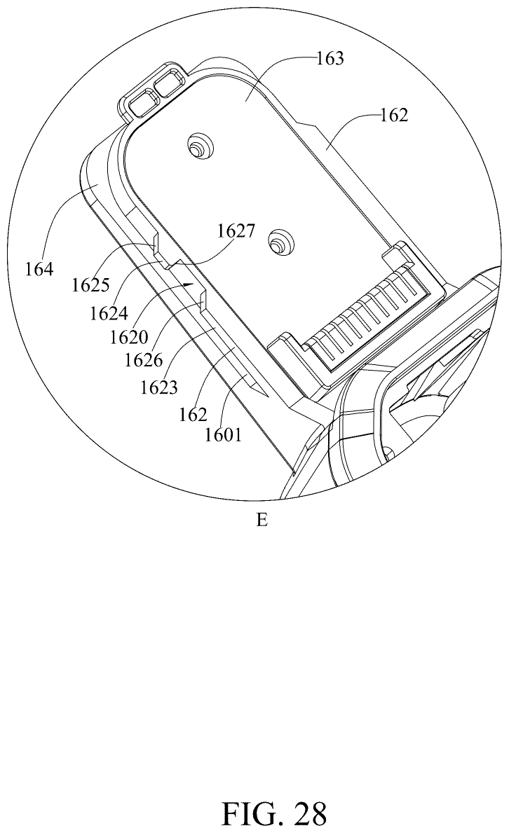

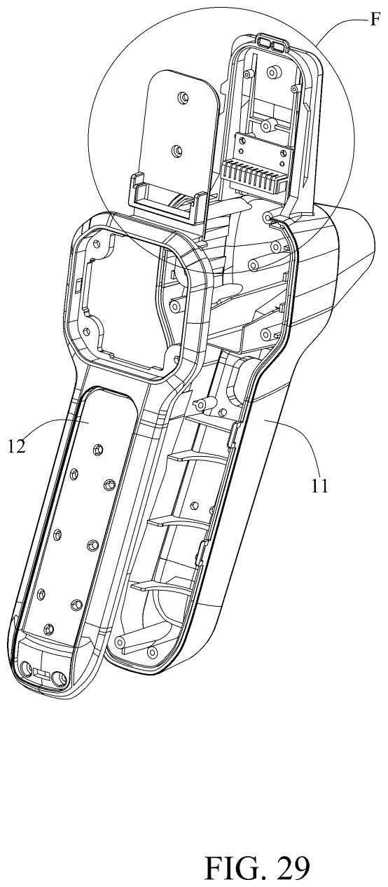

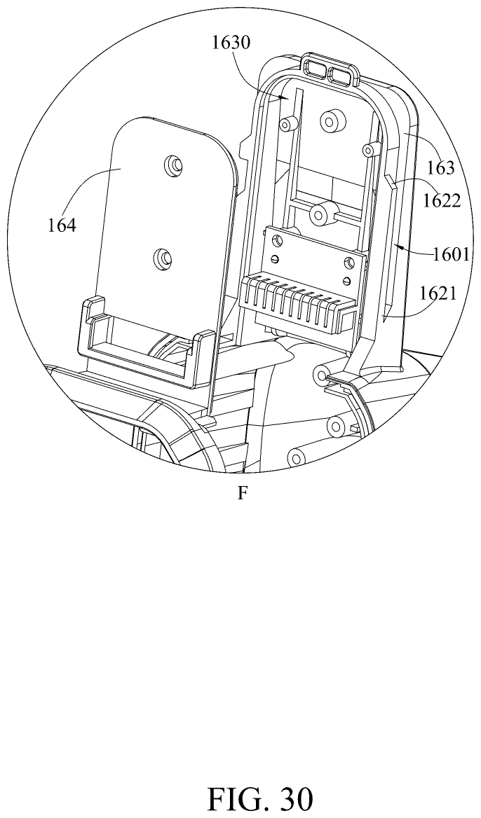

As illustrated in FIG. 1, FIG. 18 to FIG. 22, and FIG. 27 to FIG. 31, in some embodiments, the operating handle 100 has a housing 1, which includes a first housing 11 and a second housing 12 connected to the first housing 11. A connector 16 is connected to the housing 1.

In some embodiments, the connector 16 also includes a connecting member 163 and a cover 164. The connecting member 163 is connected to the first housing 11. The connecting member 163 has a mounting cavity 1630, and a fixed end of a first connection terminal 161 is fixedly installed on an inner wall of the mounting cavity 1630. The cover 164 is connected to the second housing 12. When the first housing 11 is connected to the second housing 12, the cover 164 engages with the connecting member 163 to close the mounting cavity 1630. The cover 164 is defined with an avoidance opening 1640 in communication with the mounting cavity 1630. A connection end of the first connection terminal 161 extends through the avoidance opening 1640, thereby engaging with the second connection terminal 82 when the connection head 16 is assembled with the connecting groove 801. The connecting member 163 and the cover 164 are secured by fastening screws, facilitating the installation of the first connection terminal 161.

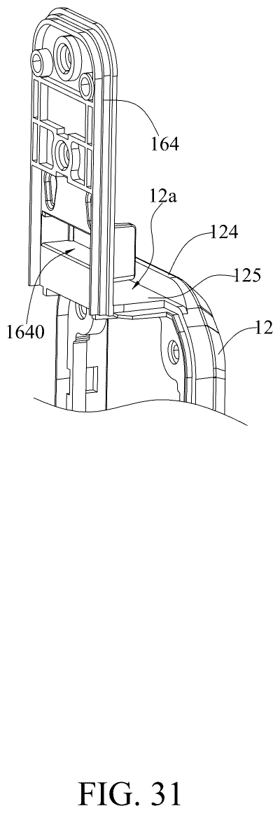

As illustrated in FIG. 19, FIG. 23 and FIG. 31, in some embodiments, the second housing 12 has a protrusion 124 and an extension 125. A first end of the protrusion 124 is connected to the second housing 12, and a second end of the protrusion 124 extends to a side away from the second housing 12. A first end of the extension 125 is connected to the first end of the protrusion 124. A second end of the extension 125 extends toward one side of the first housing 11, and is connected to the cover 164. The protrusion 124, the extension 125, and the cover 164 surround to form a recess 12a. When the display screen 800 is assembled with the operating handle 100, a portion of the assembly side 80a of the display screen 800 is accommodated in the recess 12a, securing the display screen 800 and further improving the stability of the connection between the display screen 800 and the operating handle 100.

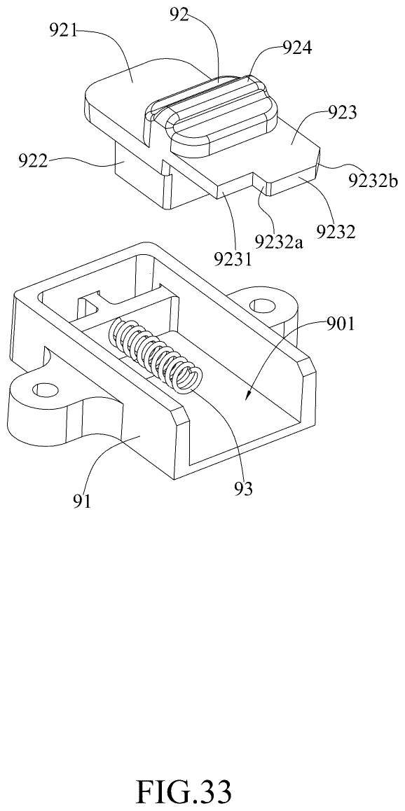

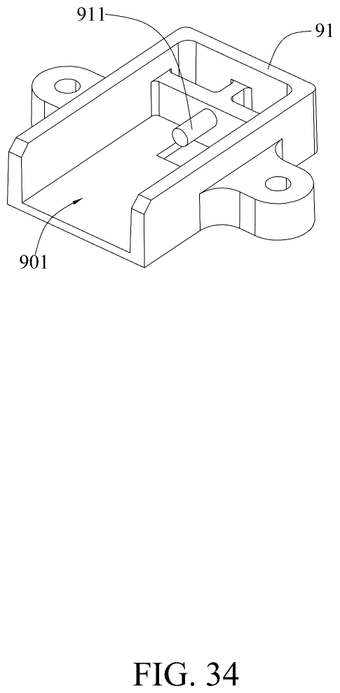

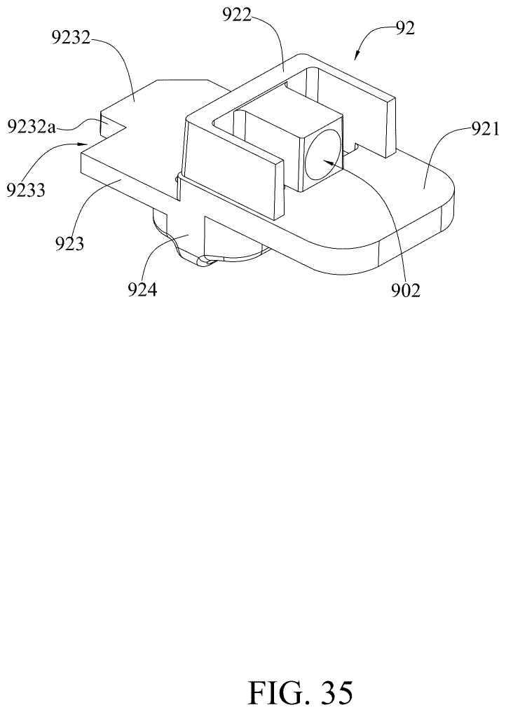

As illustrated in FIG. 1, FIG. 18 to FIG. 26 and FIG. 32 to FIG. 35, in some embodiments, the snap-fit assembly 900 includes a seat 91, a snap-fit member 92 and a second elastic member 93. In some embodiments, the seat 91 is disposed in the inner cavity 802 of the casing 81 and is fixedly connected to an inner wall of the rear cover 812 by a fastening screw. The snap-fit member 92 is movably disposed in the snap-fit channel 803. A second end of the snap-fit member 92 extends into the inner cavity 802 of the casing 81, and a first end of the snap-fit member 92 can be selectively engaged or disengaged with the connector 16, realizing the locking and unlocking between the connector 16 and the connecting groove 801. The second elastic member 93 is arranged between a second end of the snap-fit member 92 and the seat 91. A first end of the second elastic member 93 is connected to the seat 91, and a second end of the second elastic member 93 is connected to the second end of the snap-fit member 92. The second elastic member 93 is configured to squeeze the snap-fit member 92 into the first engaging groove 805, allowing the snap-fit member 92 to extend out of the snap-fit channel 803 and to clamp with the connector 16.

In some embodiments, the setting of the snap-fit assembly 900 can improve the disassembly and assembly between the display screen 800 and the operating handle 100, facilitating the packaging, transportation and carrying of the endoscope. In addition, the setting of the snap-fit assembly 900 also ensures the stability of the connection between the display screen 800 and the operating handle 100, and reduces the occurrences of the display screen 800 from being detached from the operating handle 100 during use.

In some embodiments, the rear cover 812 is defined with a receiving port 806 in communication with the snap-fit channel 803. The seat 91 is defined with a second mounting groove 901. The snap-fit member 92 includes a retention portion 921, a second mounting portion 922, an engaging portion 923 and a driving portion 924.

In some embodiments, the retention portion 921 is accommodated in the second mounting groove 901. The retention portion 921 is located in the inner cavity 802 and fits with the inner wall of the rear cover 812 to restrict the position of the snap-fit member 92. The second mounting portion 922 is extended on an end surface of the retention portion 921 proximal to the bottom of the second mounting groove 901, and is connected to a second end of the second elastic member 93. The engaging portion 923 is connected to an end surface of the retention portion 921 away from a bottom of the second mounting groove 901. The engaging portion 923 is accommodated in the snap-fit channel 803 and is capable of moving along the snap-fit channel 803 to extend out of or retract into the snap-fit channel 803. The driving portion 924 is disposed in the receiving port 806 and is connected to the engaging portion 923. The driving portion 924 is configured for a user to apply force to drive the engaging portion 923 to retract into the snap-fit channel 803, thereby manually controlling the connection head 16 to detach from the connecting groove 801.

In some embodiments, when manually controlling the snap-fit member 92, since the driving portion 924 is accommodated in the receiving port 806, a user can directly apply external force to the driving portion 924, forcing the driving portion 924 to drive the engaging portion 923, the second mounting portion 922 and the retention portion 921 to move synchronously, and compress the second elastic member 93. In this way, the snap-fit member 92 is detached from the connector 16 and retracts into the snap-fit channel 803. After the external force is removed, the snap-fit member 92 is reset under the elastic force of the second elastic member 93, allowing the snap-fit member 92 to be automatically clamped with the connector 16 under the elastic force of the second elastic member 93 when the display screen 800 is connected to the operating handle 100 again. Such a setting makes it easier to assemble or detach the connector 16 from the connecting groove 801, and effectively improves the efficiency of disassembling and assembling the display screen 80 and the operating handle 100.

In some embodiments, the driving portion 924 may be connected only to the engaging portion 923, may be adjacent only to the retention portion 921, or may be connected to both the engaging portion 923 and the retention portion 921. It only needs to satisfy the requirement that the engaging portion 923, the retention portion 921 and the second mounting portion 922 can be driven to move synchronously by applying external force to the driving portion 924. In some embodiments, the driving portion 924 is integrally formed with the engaging portion 923 and the retention portion 921.

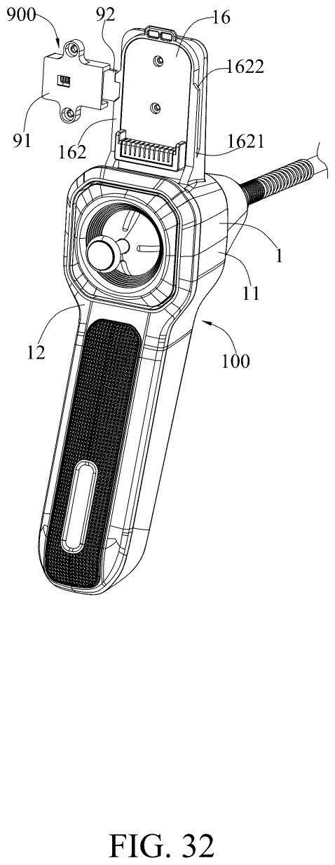

As illustrated in FIG. 32 to FIG. 35, in some embodiments, the second mounting portion 922 is defined with a second mounting hole 902. The second end of the second elastic member 93 extends into the second mounting hole 902 and is connected to a bottom of the second mounting hole 902. A connecting column 911 is provided on an end surface of the second mounting groove 901 opposite to the second mounting hole 902, and the first end of the second elastic member 93 is sleeved on the connecting column 911.

In some embodiments, the first end of the second elastic member 93 is sleeved on the connecting column 911. When elastic deformation occurs, the connecting column 911 plays a guiding role, thereby avoiding the accidental deformation of the second elastic member 93 or the slipping of the first end during the elastic deformation, and ensuring the deformation of the second elastic member 93. In addition, the second end of the second elastic member 93 is disposed in the second mounting hole 902. The second mounting hole 902 also plays a guiding role when the second elastic member 93 undergoes elastic deformation, and restrict the second end of the second elastic member 93, thereby further ensuring the deformation of the second elastic member 93. In some embodiments, the snap-fit channel 803 extends along a third direction Y, and the second elastic member 93 undergoes elastic deformation along the third direction Y.

In some embodiments, the connecting column 911 is arranged on the second mounting portion 922, and correspondingly, the second mounting hole 902 is defined by the sidewall of the second mounting groove 901. In some embodiments, a connecting column 911 is provided on each of the second mounting portion 922 and the sidewall of the second mounting groove 901, and the first end and the second end of the second elastic member 93 are respectively sleeved on the two connecting columns 911. In some embodiments, a second mounting hole 902 is defined by each of the second mounting portion 922 and the sidewall of the second mounting groove 901, and the first end and the second end of the second elastic member 93 are respectively disposed in the two second mounting holes 902. In some embodiments, the first end of the second elastic member 93 is fixed to the inner wall of the second mounting groove 901, and the second end of the second elastic member 93 is fixed to the second mounting portion 922. In other words, it is only necessary to connect the first end of the second elastic member 93 with the seat 91, connect the second end of the second elastic member 93 with the snap-fit member 92, and make the second elastic member 93 provide elastic force for the snap-fit member 92 to extend out of the snap-fit channel 803. In some embodiments, the second elastic member 93 is a spring, and in some other embodiments, the second elastic member 93 is a spring sheet, a spring block, etc.

As illustrated in FIG. 1 and FIG. 32 to FIG. 35, in some embodiments, an end face of the engaging portion 923 proximal to the connecting groove 801 is a first end face 9231. A tab 9232 is provided on the first end face 9231, and the tab 9232 extends toward the connecting groove 801. The tab 9232 has a first limiting end face 9232a, and the first limiting end face 9232a intersects with the first end face 9231 to form a notch 9233. An engaging slot 1620 engaging with the tab 9232 is provided on the sidewall of the third retention member 162. The third retention member 162 includes two limiting strips symmetrically arranged on the outer sidewall of the connector 16, and the engaging slot 1620 separates the third retention member 162 proximal to the snap-fit assembly 900 to form a first protrusion 1623 and a second protrusion 1624. The tab 9232 fits with the engaging slot 1620 to secure the third retention member 162 in the first direction X. The second protrusion 1624 is engaged with the notch 9233.

In some embodiments, an end of the tab 9232 away from the first limiting end face 9232a is defined with a wedge-shaped end face 9232b. A head end of the first protrusion 1623 has a first inclined end face 1625. A head end of the second protrusion 1624 has a second inclined end face 1626, and a bottom end of the second protrusion 1624 has a second limiting end face 1627. The inclination directions of the wedge-shaped end face 9232b, the first inclined end face 1625 and the second inclined end face 1626 are the same.

In some embodiments, during the engagement between the third retention member 162 and the first engaging groove 805, the first inclined end surface 1625 is fit with the wedge-shaped end surface 9232b, and the tab 9232 is squeezed into the snap-fit channel 803, so that the tab 9232 is retracted into the snap-fit channel 803 and the second elastic member 93 is compressed synchronically. When the tab 9232 moves to the engaging slot 1620 to be snapped with the engaging slot 1620 under the elastic force of the second elastic member 93, and the second inclined end surface 1626 and the wedge-shaped end surface 9232b are proximal to each other, and the tab 9232 is completely accommodated.

In some embodiments, when the connector 16 and the connecting groove 801 are assembled with each other, the head end 1622 of the third retention member 162 is aligned with the first end 8051 of the first engaging groove 805, and the connector 16 is pushed along the first direction X to move the head end 1622 of the third retention member 162 toward the second end 8052 of the first engaging groove 805. When the first inclined end face 1625 abuts against the wedge-shaped end face 9232b, the connector 16 is continued to be pushed along the first direction X. The interaction between the first inclined end face 1625 and the wedge-shaped end face 9232b creates a force for squeezing the tab 9232 to move into the snap-fit channel 803, allowing the tab 9232 to move into the snap-fit channel 803 and compress the second elastic member 93. The connector 16 is further pushed along the first direction X until the tab 9232 fits with the engaging slot 1620 under the elastic force of the second elastic member 93. Then, the pushing of the connector 16 is stopped. In this way, the first limiting end face 9232a abuts against the second limiting end face 1627, and the second inclined end face 1626 fits against the wedge-shaped end face 9232b. Therefore, the first engaging groove 805 and the third retention member 162 are engaged.

From the above description, it can be seen that the above-mentioned embodiments of the present disclosure achieve the following technical effects.

1) The first elastic member 25 is arranged between the support column 23 and the mounting seat 21. After the joystick mechanism 2 is assembled, the first elastic member 25 is in a compressed state, squeezing the support column 23 to the side away from the mounting seat 21 by its own restoring force. Thus, the groove wall of the first mounting groove 230 is always in tight fit with the ball head 241, increasing the friction force of the ball head 241 relative to the first mounting groove 230, and allowing the head joint 24 to have a damping force during operation. On the one hand, it improves the operating feel of the joystick mechanism. On the other hand, the setting of the first elastic member 25 makes the support column 23 press the ball head 241 against the first retention portion 221. Therefore, the ball head 241 is stably and firmly secured between the support column 23 and the first retention portion 221, reducing the problems of loosening of the ball head 241 due to frequent operation, and improving the accuracy of the bending operation of the industrial endoscope.

2) The display screen 800 and the operating handle 100 are detachably connected, facilitating easy disassembly of the display screen 800 from the operating handle 100, thereby reducing the dimension and facilitating packaging, transportation or carrying. The snap-fit assembly 900 enables the display screen 800 and the operating handle 100 to be quickly disassembled and assembled, not only improving the stability of the connection between the display screen 800 and the operating handle 100, but also facilitating the disassembly, assembly, use, transportation or carrying of the endoscope.

The above description only illustrates some embodiments of the present disclosure, and does not limit the protection scope of the present disclosure. All equivalent structural changes made by using the contents in the specification and drawings under the concept of the present disclosure, or directly/indirectly used in other related technical fields are included in the protection scope of the present disclosure.

What is claimed is:

1. A joystick mechanism, comprising:
a mounting seat;
an outer sleeve connected to the mounting seat, and one end of the outer sleeve being provided with a first retention portion;
a support column disposed in a cavity of the outer sleeve, the support column configured to move along an axial direction of the cavity, and one end of the support column defining a first mounting groove;
a head joint having a ball head, the ball head being disposed in the first mounting groove and secured between the support column and the first retention portion, the ball head configured to move in the first mounting groove, and an end of the head joint extending out of the outer sleeve;
a first elastic member arranged in the cavity of the outer sleeve, a first end of the first elastic member being connected to the support column, a second end of the first elastic member being connected to the mounting seat, and the first elastic member configured to squeeze the support column to be away from the mounting seat; and
a joystick assembly connected to a portion of the head joint, the portion of the head joint extending out of the outer sleeve, and the joystick assembly configured to control the head joint to move in the first mounting groove.

2. The joystick mechanism according to claim 1, wherein another end of the support column is provided with a reduced portion, and the first elastic member is sleeved on the reduced portion.

3. The joystick mechanism according to claim 2, wherein the mounting seat comprises:
a seat body having a mounting boss, the seat body defining an assembly groove, the assembly groove extending through the mounting boss, and a portion of the outer sleeve being disposed in the assembly groove; and
a bottom plate detachably connected to a side of the seat body to secure the outer sleeve in the assembly groove.

4. The joystick mechanism according to claim 3, wherein a second retention portion is provided at one end of the mounting boss, and one end of the outer sleeve is provided with an enlarged portion, and the enlarged portion is secured in the assembly groove by the second retention portion and the bottom plate.

5. The joystick mechanism according to claim 3, wherein the bottom plate is defined with a through hole, and the reduced portion comprises a first portion and a second portion connected to each other, the second portion of the reduced portion extends through the through hole, and a radial dimension of the first portion of the reduced portion is greater than a radial dimension of the through hole.

6. The joystick mechanism according to claim 1, wherein the joystick assembly comprises:
a linkage member connected to another portion of the head joint, the other portion of the head joint extending through the outer sleeve;
an operating rod having an operating end and an assembly end, the assembly end extending through the linkage member, the operating rod having a channel axially extending through the operating end and the assembly end; and
a fastener having a connecting end, the connecting end extending through the channel to be connected to the head joint, the fastener configured to lock the operating rod and the linkage member to allow the operating rod, the linkage member and the head joint to move synchronously.

7. An operating handle comprising:
a housing; and
a joystick mechanism comprising:
a mounting seat;
an outer sleeve connected to the mounting seat, and one end of the outer sleeve being provided with a first retention portion;
a support column disposed in a cavity of the outer sleeve, the support column configured to move along an axial direction of the cavity, and one end of the support column defining a first mounting groove;
a head joint having a ball head, the ball head being disposed in the first mounting groove and secured between the support column and the first retention portion, the ball head configured to move in the first mounting groove, and an end of the head joint extending out of the outer sleeve;
a first elastic member arranged in the cavity of the outer sleeve, a first end of the first elastic member being connected to the support column, a second end of the first elastic member being connected to the mounting seat, and the first elastic member configured to squeeze the support column to be away from the mounting seat; and
a joystick assembly connected to a portion of the head joint, the portion of the head joint extending out of the outer sleeve, and the joystick assembly configured to control the head joint to move in the first mounting groove;
the joystick assembly comprising:
a linkage member connected to another portion of the head joint, the other portion of the head joint extending through the outer sleeve;
an operating rod having an operating end and an assembly end, the assembly end extending through the linkage member, the operating rod having a channel axially extending through the operating end and the assembly end; and
a fastener having a connecting end, the connecting end extending through the channel to be connected to the head joint, the fastener configured to lock the operating rod and the linkage member to allow the operating rod, the linkage member and the head joint to move synchronously;
wherein a portion of the joystick mechanism is arranged in the housing and connected to an inner wall of the housing by the mounting seat, and the operating end of the operating rod extends out of the housing;
a lens assembly connected to the housing, a proximal end of the lens assembly extending into the housing and connected to the linkage member, and a distal end of the lens assembly extending away from the housing; and
a protective coupling assembly sleeved on the proximal end of the lens assembly and connected to the housing.

8. The operating handle according to claim 7, wherein the lens assembly comprises a lens tube and a traction line, the traction line extends through the lens tube, a distal end of the traction line is connected to a distal end of the lens tube, a proximal end of the traction line extends through a proximal end of the lens tube and is connected to the linkage member, the operating rod is configured to control the head joint move relative to the support column, drive the linkage member to move, and control the traction line to move, thereby controlling a bending of the distal end of the lens tube.

9. The operating handle according to claim 8, wherein a linkage end is provided on an outer wall of the linkage member, the linkage end is provided with a slot, a sidewall of the slot is defined with a mounting opening, a proximal end of the traction line is connected to a fixing block, a traction line is configured to enter the slot through the mounting opening, and is secured within the slot by the fixing block.

10. The operating handle according to claim 8, wherein the protective coupling assembly comprises:
a connecting member disposed in the housing and sleeved on the proximal end of the lens tube, the connecting member has a connecting section and an assembly section, the connecting section fits with the inner wall of the housing to secure the connecting member in the housing, and the assembly section extends out of the housing;
a locking member sleeved on the proximal end of the lens tube and assembled with the assembly section to allow the locking member and the connecting section to clamp the housing; and
a winding member wound on an outer wall of the proximal end of the lens tube, and a proximal end of the winding member being connected to the connecting member.

11. An endoscope, comprising:
An operating handle comprising:
a housing; and
a joystick mechanism comprising:
a mounting seat;
an outer sleeve connected to the mounting seat, and one end of the outer sleeve being provided with a first retention portion;
a support column disposed in a cavity of the outer sleeve, the support column configured to move along an axial direction of the cavity, and one end of the support column defining a first mounting groove;
a head joint having a ball head, the ball head being disposed in the first mounting groove and restricted between the support column and the first retention portion, the ball head configured to move in the first mounting groove, and an end of the head joint extending out of the outer sleeve;
a first elastic member arranged in the cavity of the outer sleeve, a first end of the first elastic member being connected to the support column, a second end of the first elastic member being connected to the mounting seat, and the first elastic member configured to squeeze the support column to a side away from the mounting seat; and
a joystick assembly connected to a portion of the head joint, the portion of the head joint extending out of the outer sleeve, the joystick assembly being configured to control the head joint to allow the head joint to move in the first mounting groove;
the joystick assembly comprising:
a linkage member connected to another portion of the head joint, the other portion of the head joint extending through the outer sleeve;
an operating rod having an operating end and an assembly end, the assembly end extending through the linkage member, the operating rod having a channel axially extending through the operating end and the assembly end; and
a fastener having a connecting end, the fastener extending through the channel, the connecting end extending through the channel to be connected to the head joint, locking the operating rod and the linkage member to allow the operating rod, the linkage member and the head joint to move synchronously;
wherein a portion of the joystick mechanism is arranged in the housing and connected to an inner wall of the housing by the mounting seat, and the operating end of the operating rod extending out of the housing;
a lens assembly connected to the housing, a proximal end of the lens assembly extending into the housing and connected to the linkage member, and a distal end of the lens assembly extending away from the housing; and
a protective coupling assembly sleeved on the proximal end of the lens assembly and connected to the housing;
wherein the housing of the operating handle is provided with a connector, and a first connecting terminal is provided on the connector;
a display screen detachably connected to the operating handle, and the display screen comprising a casing and a second connecting terminal, the casing defining a connecting groove, the connector being inserted into the connecting groove to detachably assemble the operating handle and the display screen, a fixed end of the second connecting terminal being assembled in an inner cavity of the casing, an insertion end of the second connecting terminal extending through the casing and extending into the connecting groove, and the second connecting terminal is configured to be plugged into the first connecting terminal to conduct the operating handle and the display screen, and a sidewall of the connecting groove defining a snap-fit channel communicating with the inner cavity of the casing; and
a snap-fit assembly comprising a first portion and a second portion, the first portion of the snap-fit assembly being fixedly disposed in the inner cavity of the casing, the second portion of the snap-fit assembly being accommodated in the snap-fit channel and connected to the first portion of the snap-fit assembly, the second portion of the snap-fit assembly is configured to move relative to the snap-fit channel and extend out of the snap-fit channel to snap fit with the connector.

12. The endoscope according to claim 11, wherein the casing comprises a front casing and a rear cover fixedly assembled with the front casing, an outer wall of the rear cover defines the connecting groove, and the connecting groove defines an opening for receiving the connector;
a sidewall of the connecting groove is defined with a first engaging groove extending in a first direction, a first end of the first engaging groove is in communication with the opening; a sidewall of the connector is provided with a third retention member, and the third retention member is inserted into the first engaging groove to allow the connecting groove to secure the connector in a second direction.

13. The endoscope according to claim 12, wherein the third retention member has a head end facing the connecting groove and a bottom end opposite to the head end, and a cross-sectional dimension of the bottom end of the third retention member is greater than a cross-sectional dimension of the head end of the third retention member, a size of an opening of the first end of the first engaging groove is greater than a size of an opening of the second end of the first engaging groove.

14. The endoscope according to claim 12, wherein the sidewall of the connecting groove is provided with a fourth retention portion, the sidewall of the connector is defined with a second engaging groove, and the fourth retention portion is inserted into the second engaging groove.

15. The endoscope according to claim 11, wherein the snap-fit assembly fits with the connector to secure the connector in the first direction, and the snap-fit assembly comprises:
a seat disposed in the inner cavity of the casing and fixedly connected to an inner wall of the rear cover;
a snap-fit member movably disposed in the snap-fit channel, a second end of the snap-fit member extending into the inner cavity of the casing, and a first end of the snap-fit member is configured to selectively engage or disengage with the connector; and
a second elastic member arranged in the inner cavity of the casing and positioned between the second end of the snap-fit member and the seat, a first end of the second elastic member being connected to the seat, a second end of the second elastic member being connected to the second end of the snap-fit member, and the second elastic member is configured to squeeze the snap-fit member into the first engaging groove and extend out of the snap-fit channel to be snapped with the connector.

16. The endoscope according to claim 15, wherein the rear cover is provided with a receiving port in communication with the snap-fit channel, the seat is defined with a second mounting groove, and the snap-fit member comprises:
a retention portion accommodated in the second mounting groove, and the retention portion engaging with the inner wall of the rear cover to secure the snap-fit member;
a second mounting portion protruded on an end surface of the retention portion facing the bottom of the second mounting groove, and the second end of the second elastic member being connected to the second mounting portion;
an engaging portion connected to the retention portion, the engaging portion accommodated in the snap-fit channel and configured to move along the snap-fit channel; and
a driving portion disposed in the receiving port and connected to the engaging portion or/and the retention portion, and the driving portion configured to drive the engaging portion to retract into the snap-fit channel.

17. The endoscope according to claim 16, wherein the second mounting portion is defined with a second mounting hole, and the second end of the second elastic member extends into the second mounting hole to be connected to a bottom of the second mounting hole; or/and
a connecting column is protruded on an end surface of the second mounting groove opposite to the second mounting hole, and the first end of the second elastic member is sleeved on the connecting column and connected to an inner wall of the second mounting groove.

18. The endoscope according to claim 16, wherein the engaging portion comprises a first end surface proximal to the connecting groove, and a tab is provided on the first end surface; a sidewall of the third retention member is defined with an engaging slot for engaging with the tab, and the tab is configured to selectively extend into the engaging slot to secure the third retention member in the first direction.

19. The endoscope according to claim 11, wherein the operating handle comprises a housing, the housing comprises a first housing and a second housing, the connector comprises:
a connecting member connected to the first housing, the connecting member defining a mounting cavity, and a fixed end of the first connecting terminal being fixedly mounted on an inner wall of the mounting cavity; and
a cover connected to the second housing, the cover engaging with the connecting member to close the mounting cavity in response to the first housing being connected to the second housing, the cover defining an avoidance opening communicating with the mounting cavity, and a connecting end of the first connecting terminal extending through the avoidance opening to engage with the second connecting terminal.

20. The endoscope according to claim 19, wherein the second housing comprises a protrusion and an extension, a first end of the protrusion is connected to the second housing, a second end of the protrusion extends to a side away from the second housing; a first end of the extension is connected to the first end of the protrusion, a second end of the extension extends to a side of the first housing and is connected to the cover; the cover, the extension portion and the protrusion are surrounded to form a recess for receiving the casing of the display screen.
